SEQUENCE LISTING (34 sequences)

SEQ ID NO: 1 - protein, 4 aa, Artificial Sequence
  ELP component sequence
VPGG

SEQ ID NO: 2 - protein, 4 aa, Artificial Sequence
  ELP component sequence
IPGG

SEQ ID NO: 3 - protein, 5 aa, Artificial Sequence
  ELP component sequence
VPGXG

SEQ ID NO: 4 - protein, 5 aa, Artificial Sequence
  ELP component sequence
AVGVP

SEQ ID NO: 5 - protein, 5 aa, Artificial Sequence
  ELP component sequence
IPGXG

SEQ ID NO: 6 - protein, 5 aa, Artificial Sequence
  ELP component sequence
IPGVG

SEQ ID NO: 7 - protein, 5 aa, Artificial Sequence
  ELP component sequence
LPGXG

SEQ ID NO: 8 - protein, 5 aa, Artificial Sequence
  ELP component sequence
LPGVG

SEQ ID NO: 9 - protein, 6 aa, Artificial Sequence
  ELP component sequence
VAPGVG

SEQ ID NO: 10 - protein, 8 aa, Artificial Sequence
  ELP component sequence
GVGVPGVG

SEQ ID NO: 11 - protein, 9 aa, Artificial Sequence
  ELP component sequence
VPGFGVGAG

SEQ ID NO: 12 - protein, 9 aa, Artificial Sequence
  ELP component sequence
VPGVGVPGG

SEQ ID NO: 13 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 14 - protein, 691 aa, Artificial Sequence
  ELP-1-120 fusion protein
FVNQHLCGSHLVEALYLVCGERGFFYTPKTRREAEDLQVGQVELGGGPGAGSLQPLALEGSLQKRGIVEQCCTSICSLYQLENYCNVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 15 - protein, 21 aa, Homo sapiens
GIVEQCCASVCSLYQLENYCN

SEQ ID NO: 16 - protein, 30 aa, Homo sapiens
FVNQHLCGSHLVEALYLVCGERGFFYTPKA

SEQ ID NO: 17 - protein, 12 aa, Artificial Sequence
  Linker peptide
KDDNPNLPRLVR

SEQ ID NO: 18 - protein, 12 aa, Artificial Sequence
  Linker peptide
GAGSSSRRAPQT

SEQ ID NO: 19 - DNA, 261 bp, Homo sapiens
atgtttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaac

SEQ ID NO: 20 - protein, 87 aa, Homo sapiens
MFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 21 - protein, 4 aa, Artificial Sequence
  Linker peptide
GFLG

SEQ ID NO: 22 - protein, 37 aa, Homo sapiens
HDEFERHAEGTFTSDVSSYLEGQAAKEFIAWLVKGRG

SEQ ID NO: 23 - protein, 87 aa, Heloderma suspectum
MKIILWLCVFGLFLATLFPISWQMPVESGLSSEDSASSESFASKIKRHGEGTFTSDLSKQMEEEAVRLFIEWLKNGGPSSGAPPPSG

SEQ ID NO: 24 - protein, 30 aa, Homo sapiens
HAEGTFTSDVSSYLEGQAAKEFIAWLVKGR

SEQ ID NO: 25 - protein, 31 aa, Homo sapiens
HAEGTFTSDVSSYLEGQAAKEFIAWLVKGRG

SEQ ID NO: 26 - protein, 33 aa, Homo sapiens
HADGSFSDEMNTILDNLAARDFINWLIQTKITD

SEQ ID NO: 27 - protein, 153 aa, Homo sapiens
MVATKTFALLLLSLFLAVGLGEKKEGHFSALPSLPVGSHAKVSSPQPRGPRYAEGTFISDYSIAMDKIHQQDFVNWLLAQKGKKNDWKHNITQREARALELASQANRKEEEAVEPQSSPAKNPSDEDLLRDLLIQELLACLLDQTNLCRLRSR

SEQ ID NO: 28 - protein, 180 aa, Homo sapiens
MKSIYFVAGLFVMLVQGSWQRSLQDTEEKSRSFSASQADPLSDPDQMNEDKRHSQGTFTSDYSKYLDSRRAQDFVQWLMNTKRNRNNIAKRHDEFERHAEGTFTSDVSSYLEGQAAKEFIAWLVKGRGRRDFPEEVAIVEELGRRHADGSFSDEMNTILDNLAARDFINWLIQTKITDRK

SEQ ID NO: 29 - protein, 37 aa, Homo sapiens
HSQGTFTSDYSKYLDSRRAQDFVQWLMNTKRNRNNIA

SEQ ID NO: 30 - protein, 300 aa, Artificial Sequence
  ELP biopolymer sequence
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 31 - protein, 450 aa, Artificial Sequence
  ELP biopolymer sequence
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 32 - protein, 600 aa, Artificial Sequence
  ELP biopolymer sequence
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 33 - protein, 900 aa, Artificial Sequence
  ELP biopolymer sequence
VPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXGVPGXG

SEQ ID NO: 34 - protein, 20 aa, Artificial Sequence
  non-cleavable spacer peptide
GGGGSGGGGSGGGGSGGGGS

(19) United States
(12) Patent Application Publication
(10) Pub. No.: US 2013/0150291 A1
(43) Pub. Date: Jun. 13, 2013

(54) THERAPEUTIC AGENTS COMPRISING INSULIN AMINO ACID SEQUENCES

(71) Applicant: PhaseBio Pharmaceuticals, Inc., Malvern, PA (US)

(72) Inventors: James Jowett, Malvern, PA (US); Christopher Woods, Malvern, PA (US)

(73) Assignee: PHASEBIO PHARMACEUTICALS, INC., Malvern, PA (US)

(21) Appl. No.: 13/687,776

(22) Filed: Nov. 28, 2012

Related U.S. Application Data
(60) Provisional application No. 61/563,985, filed on Nov. 28, 2011.

(51) Int. Cl. A61K 9/00

(52) U.S. Cl. CPC A61K 9/0002
USPC 514/5.9

(57) ABSTRACT

The present invention relates in part to agents which provide slow absorption from an injection site. In some embodiments, the pharmaceutical compositions comprises an insulin amino acid sequence and an amino acid sequence that provide slow absorption from an injection site, such as, for example, an amino acid sequence that has a substantially repeating pattern of proline residues.

### PRIORITY

This application claims priority from U.S. Provisional Application No. 61/563,985, filed Nov. 28, 2011, the contents of which are incorporated by reference in their entirety.

### FIELD OF THE INVENTION

The present invention relates in part to forms of insulin and derivatives thereof with sustained biological action.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: PHAS_{—}025_{—}01US_SeqList_ST25.txt, date recorded: Nov. 28, 2012, file size 38 kilobytes).

### BACKGROUND

The effectiveness of peptide and small molecule drugs is often limited by the half-life of such drugs in the circulation, as well as difficulties in obtaining substantially constant plasma levels. For example, the incretin GLP-1 must be administered at relatively high doses to counter its short half-life in the circulation, and these high doses are associated with nausea, among other things. Murphy and Bloom, Nonpeptidic glucagon-like peptide 1 receptor agonists: A magic bullet for diabetes? PNAS 104 (3):689-690 (2007). Further, the peptide agent vasoactive intestinal peptide (VIP) exhibits a half-life, in some estimates, of less than one minute, making this agent impractical for pharmaceutical use. Domschke et al., Vasoactive intestinal peptide in man: pharmacokinetics, metabolic and circulatory effects, Gut 19:1049-1053 (1978); Henning and Sawmiller, Vasoactive intestinal peptide: cardiovascular effects, Cardiovascular Research 49:27-37 (2001). A short plasma half life for peptide drugs is often due to fast renal clearance as well as to enzymatic degradation during systemic circulation.

Insulin, or derivatives thereof, suffer from similar difficulties. Insulin is active for only a brief time before it is degraded by enzymes (e.g. insulinase) and therefore has a half-life of only about 6 minutes. Also, insulin may be absorbed quickly by a subject and therefore such a subject may require two or more injections of insulin daily, with doses adjusted on the basis of self-monitoring of blood glucose levels. Further, spike and troughs in insulin levels create significant complications for subjects. There remains a need for insulin therapies that display slow absorption into the circulation and provide an extended steady state level of glucose control.

### SUMMARY OF THE INVENTION

The present invention provides insulin-based pharmaceutical formulations for sustained release, and methods for delivering a treatment regimen with the sustained release formulations. The invention thereby provides improved pharmacokinetics for insulin-based pharmaceutical formulations.

In one aspect, the invention provides a pharmaceutical composition for providing sustained glycemic control comprising an effective amount of a protein, the protein comprising an insulin amino acid sequence and an amino acid sequence providing a sustained release from an injection site, and pharmaceutical excipients to achieve sustained release.

In another aspect, the invention provides methods of treating diabetes involving administering a pharmaceutical composition for providing sustained glycemic control. The composition comprises an effective amount of a protein comprising an insulin amino acid sequence and an amino acid sequence providing a sustained release from an injection site, and pharmaceutical excipients to achieve sustained release to a patient in need thereof. In some embodiments, the patient has type 1 diabetes or type 2 diabetes. In some embodiments, the method comprises administering the pharmaceutical composition at a frequency of from 1 to about 30 times per month, or about weekly, or about two or three times per week, or about daily. In some embodiments, the method comprises administering the pharmaceutical composition subcutaneously.

### BRIEF DESCRIPTION OF THE FIGURES

FIG. 1A shows the human proinsulin sequence (SEQ ID NO: 13). The proinsulin sequence consists of the B and A chains linked with the C peptide. The C peptide is removed to form mature insulin following enzymatic cleavage at the two adjacent dibasic sites (underlined in italics).

FIG. 1B shows a diagram of a construction termed PE0139 or INSUMERA or Insulin-ELP1-120, having 120 ELP units fused to the C-terminus of the A chain.

FIG. 2 shows a map of the pPE0139 plasmid.

FIG. 3 shows the amino acid sequence of a proinsulin ELP1-120 fusion protein (SEQ ID NO: 14). The proinsulin sequence (underlined) is fused to the ELP1-120 sequence. The amino acid sequence optionally includes an initiation methionine residue at the N terminus.

FIG. 4 shows a non-reducing SDS-PAGE experiment. Non-reducing SDS-PAGE showed the expected decreased fusion protein molecular weight following enzymatic processing as the C-peptide was cleaved. Lane 1: SEEBLUE® Plus2 pre-stained standard (INVITROGEN), lane 2: ELP1-120, lane 3: Proinsulin ELP1-120, lane 4: Insulin ELP-120 3 μg, lane 5: Insulin ELP1-120 6 μg, lane 6: SEEBLUE® Plus2 pre-stained standard (INVITROGEN).

FIG. 5 shows an anti-insulin B chain western blot. An anti-insulin B chain western blot was performed to confirm presence of both A and B chains fused to ELP. The data showed presence of B-chain under non-reducing conditions indicating disulfide bond formation between the A and B chains. Reduction of the fusion protein and disulfide bonds resulted in removal of B chain from the fusion. Lane 1: reduced Insulin ELP fusion showing absence of B chain, lane 2: Non-reduced Insulin ELP fusion showing presence of B-chain, lane 3: ELP1-120, lane 4: Proinsulin ELP fusion showing presence of B-chain.

FIG. 6 shows ESI-MS data on unprocessed insulin-ELP1-120. Electrospray ionization mass spectrometry confirmed the mass of unprocessed Proinsulin ELP fusion of 57008.5 Da (SGS Mscan Codes 104531 & 104532). Additional salt adducts were present.

FIG. 7 shows ESI-MS data on processed pPE0139. Electrospray ionization mass spectrometry confirmed the mass of mature Insulin ELP fusion following enzymatic removal of the C-peptide (SGS M-scan Codes 107610). ESI-MS of Insulin ELP showed a main product peak with a molecular mass of approximately 53298 Da indicating mature Insulin ELP following C-peptide cleavage. Minor peaks are likely attributable as partially degraded fusion or salt adducts.

FIG. 8 shows blood glucose lowering in normal mice with Insulin-ELP1-120 fusion as compared to insulin glargine.

FIG. 9 shows INSUMERA (PE0139) dosing in a diabetes mellitus type I (type 1 diabetes, T1DM) mouse model Specifically, single dose data is shown. The results demonstrate greater duration of glucose lowering for INSUMERA, as compared to equimolar LANTUS (insulin glargine, SANOFI-AVENTIS) dosing. STZ is streptozotocin; the untreated group refers to normal, non-diabetic animals; N=8 per group.

FIG. 10 shows INSUMERA (PE01.39) dosing in a diabetes mellitus type 1 (type 1 diabetes, T1DM) mouse model. Specifically, daily dosing data is shown. The results demonstrate the superiority of INSUMERA, as compared to LANTUS (insulin glargine, SANOFI-AVENTIS), with regards to activity and half-life. STZ is streptozotocin; the untreated group refers to normal, non-diabetic animals; at the 6 h time point, N=5 for the 25 mg and 50 mg/kg groups; at the 8 h time point, N=3 for the 25 mg/kg group and n=2 for the 50 mg/kg group; at the 24 h time point, N=1 for the 25 mg/kg and N=7 for the 5 mg/kg groups.

FIG. 11A shows INSUMERA (PE0139) low dose titration in a diabetes mellitus type 1 (type 1 diabetes, T1DM) mouse model as compared to LANTUS (insulin glargine, SANOFI-AVENTIS). Specifically, FIG. 11A shows a single s.c. dose. STZ is streptozotocin; the untreated group refers to normal, non-diabetic animals; N=8 for LANTUS, PE0139 1 mg/kg and untreated groups; N=7 for the PE0139 3.33 mg/kg group.

FIG. 11B shows INSUMERA (PE0139) low dose titration in a diabetes mellitus type 1 (type 1 diabetes, T1DM) mouse model as compared to LANTUS (insulin glargine, SANOFI-AVENTIS). Specifically, FIG. 11B shows 14 days of daily s.c. dosing. STZ is streptozotocin; the untreated group refers to normal, non-diabetic animals; N=8 for LANTUS, PE0139 1 mg/kg and untreated groups; N=7 for the PE0139 3.33 mg/kg group.

FIG. 12A shows that INSUMERA (PEW 39) has significantly increased glycemic control relative to LANTUS (insulin glargine, SANOFI-AVENTIS). A reduction of 27-39% is seen in area under the curve (AUC) blood glucose on days 1, 3, 7 and 14 relative to Lantus. Specifically, FIG. 12A shows day 1 of compound administration and the blood glucose AUC at 0-24 hrs.

FIG. 12B shows that INSUMERA (PE0139) has significantly increased glycemic control relative to LANTUS (insulin glargine, SANOFI-AVENTIS). A reduction of 27-39% is seen in area under the curve (AUC) blood glucose on days 1, 3, 7 and 14 relative to Lantus. Specifically, FIG. 12B shows day 14 of compound administration and the blood glucose AUC at 0-24 hrs.

FIG. 13A shows that INSUMERA (PE0139) achieves a long half-life with a small peak to trough ratio following a subcutaneous injection, Specifically, FIG. 13A shows pharmacokinetic (PK) drug levels following a single s.c. injection in diabetic swine.

FIG. 13B shows that INSUMERA (PE0139) achieves steady state peak to trough pharmacokinetic (PK) levels following daily subcutaneous injections. Specifically, FIG. 13B shows daily s.c. injections in diabetic swine for 2 weeks; PK levels measured prior to dosing.

### DETAILED DESCRIPTION

The present invention provides insulin-based pharmaceutical compositions that exhibit sustained biological action. Also provided are methods of treating disease, including hyperglycemia and diabetes, with the compositions of the present invention.

In one aspect, the invention provides a pharmaceutical composition for providing sustained glycemic control comprising an effective amount of a protein, which comprises an insulin amino acid sequence and an amino acid sequence providing a sustained release from an injection site, and pharmaceutical excipients to achieve sustained release.

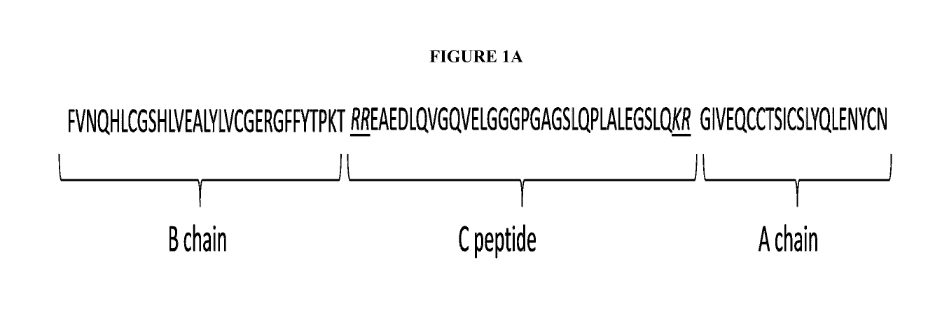

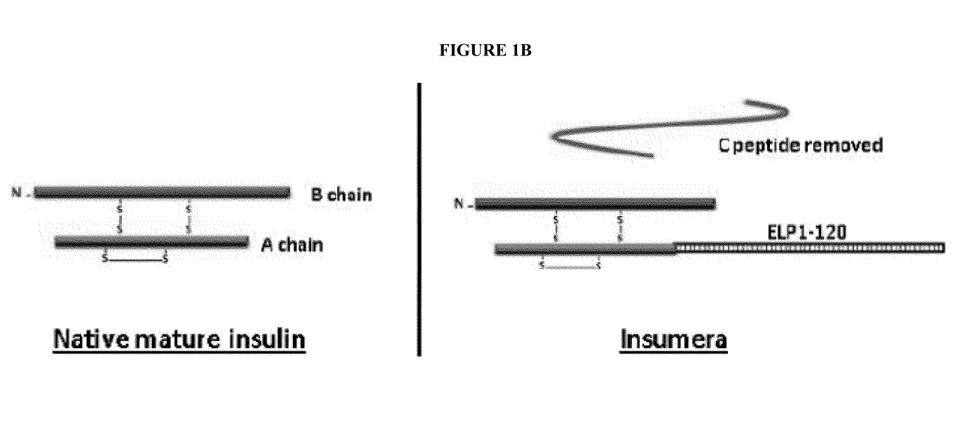

In some embodiments the insulin amino acid sequence comprises an A chain and a B chain amino acid sequence and the A chain and B chain have the amino acid sequence of SEQ ID NO: 13 (FIG. 1), optionally having from 1 to 8 amino acid insertions, deletions, or substitutions, collectively. In some embodiments, the amino acid sequence that provides a slow absorption from the injection site is covalently bound to the insulin A chain. In another embodiment, the A chain and B chain are bound by one or more disulfide bonds or attached through a peptide or chemical linker.

In another embodiment, the amino acid sequence providing a sustained release has a substantially repeating pattern of proline residues. The substantially repeating pattern may form a series or pattern of β turns. In other embodiments, the amino acid sequence providing a sustained release is an elastin-like peptide (ELP) amino acid sequence. In another embodiment, the ELP comprises repeats of VPGXG (SEQ ID NO: 3), where each X is independently selected from alanine, arginine, asparagine, aspartic acid, glutamic acid, glutamine, glycine, histidine, isoleucine, leucine, lysine, methionine, phenylalanine, serine, threonine, tryptophan, tyrosine and valine residues. In another embodiment, the ELP amino acid sequence comprises repeats of AVGVP (SEQ ID NO: 4), IPGVG (SEQ ID NO: 6), or LPGVG (SEQ ID NO: 8). In various embodiments, the ELP comprises at least 15, or at least 30, or at least 60, or at least 90, or at least 120, or at least 180 repeats of an ELP amino acid unit. In another embodiment, the ELP amino acid sequence has a transition temperature of just less than 37° C. in normal saline

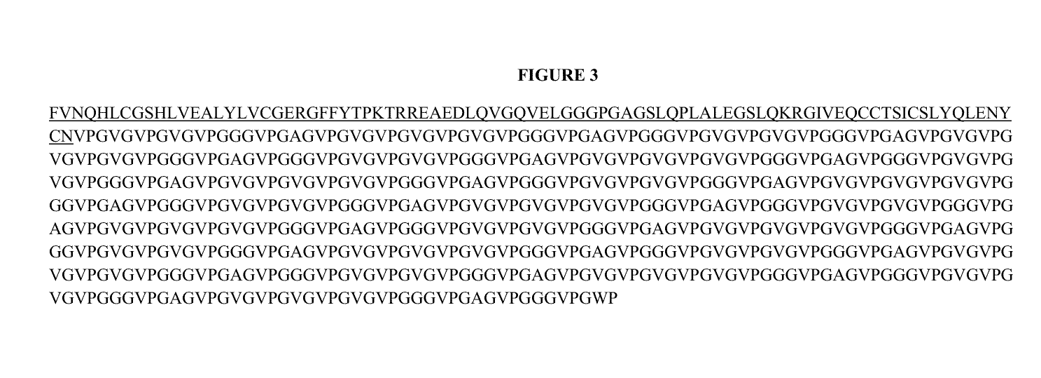

In another embodiment, the pharmaceutical composition is a fusion protein. In another embodiment, the pharmaceutical composition comprises SEQ ID NO: 14 (FIG. 3).

In yet another embodiment, the amino acid sequence providing a sustained release forms a random coil or non-globular extended structure or unstructured biopolymer, including a biopolymer where at least 50% of the amino acids are devoid of secondary structure as determined by Chou-Fasman algorithm. In yet another embodiment, the amino acid sequence providing a sustained release is a protein having an extended, non-globular structure, or a random coil structure.

In another aspect, the invention provides methods of treating diabetes involving administering the pharmaceutical composition described herein to a patient in need. In some embodiments, the patient has hyperglycemia, type 1 diabetes or type 2 diabetes, or obesity. In some embodiments, the method comprises administering the pharmaceutical composition at a frequency of from 1 to about 30 times per month, or about weekly, or about two or three times per week, or about daily. In some embodiments, the method comprises administering the pharmaceutical composition subcutaneously.

### Insulin Amino Acid Sequences

Insulin injections, e.g. of human insulin, can be used to treat diabetes. The insulin-making cells of the body are called β-cells, and they are found in the pancreas gland. These cells clump together to form the “islets of Langerhans,” named for the German medical student who described them.

The synthesis of insulin begins at the translation of the insulin gene, which resides on chromosome 11. During translation, two introns are spliced out of the mRNA product, which encodes a protein of 110 amino acids in length. This primary translation product is called preproinsulin and is inactive. It contains a signal peptide of 24 amino acids in length, which is required for the protein to cross the cell membrane. Human proinsulin consists of A and B chains linked together with the 31 amino acid C peptide (FIG. 1).

Once the preproinsulin reaches the endoplasmic reticulum, a protease cleaves off the signal peptide to create proinsulin. Specifically, once disulfide bonds are formed between the A and B chains the proinsulin is converted into mature insulin in vivo by removal of the C peptide by a trypsin/carboxypeptidase B-like system. Proinsulin consists of three domains: an amino-terminal B chain, a carboxyl-terminal A chain, and a connecting peptide in the middle known as the C-peptide. Insulin is composed of two chains of amino acids named chain A (21 amino acids—GIVEQCCASVCSLYQLENYCN) (SEQ ID NO: 15) and chain B (30 amino acids FVNQHLCGSHLVEALYLVCGERGFFYTPKA) (SEQ ID NO: 16) that are linked together by two disulfide bridges. There is a 3rd disulfide bridge within the A chain that links the 6th and 11th residues of the A chain together. In most species, the length and amino acid compositions of chains A and B are similar, and the positions of the three disulfide bonds are highly conserved. For this reason, pig insulin can replace deficient human insulin levels in diabetes patients. Today, porcine insulin has largely been replaced by the mass production of human proinsulin by bacteria (recombinant insulin).

Insulin molecules have a tendency to form dimers in solution, and in the presence of zinc ions, insulin dimers associate into hexamers. Whereas monomers of insulin readily diffuse through the blood and have a rapid effect, hexamers diffuse slowly and have a delayed onset of action. In the design of recombinant insulin, the structure of insulin can be modified in a way that reduces the tendency of the insulin molecule to form dimers and hexamers but that does not interrupt binding to the insulin receptor. In this way, a range of preparations are made, varying from short acting to long acting.

Within the endoplasmic reticulum, proinsulin is exposed to several specific peptidases that remove the C-peptide and generate the mature and active form of insulin. In the Golgi apparatus, insulin and free C-peptide are packaged into secretory granules, which accumulate in the cytoplasm of the β-cells. Exocytosis of the granules is triggered by the entry of glucose into the beta cells. The secretion of insulin has a broad impact on metabolism.

There are two phases of insulin release in response to a rise in glucose. The first is an immediate release of insulin. This is attributable to the release of preformed insulin, which is stored in secretory granules. After a short delay, there is a second, more prolonged release of newly synthesized insulin.

Once released, insulin is active for only a brief time before it is degraded by enzymes. Insulinase found in the liver and kidneys breaks down insulin circulating in the plasma, and as a result, insulin has a half-life of only about 6 minutes. This short duration of action results in rapid changes in the circulating levels of insulin.

Insulin analogs have been developed with improved therapeutic properties (Owens et al., 2001, Lancet 358: 739-46; Vajo et al., 2001, Endocr Rev 22: 706-17), and such analogs may be employed in connection with the present invention. Various strategies, including elongation of the COOH-terminal end of the insulin B-chain and engineering of fatty acid-acylated insulins with substantial affinity for albumin are used to generate longer-acting insulin analogs. However, in vivo treatments with available longer-acting insulin compounds still result in a high frequency of hypo- and hyperglycemic excursions and modest reduction in HbA1c. Accordingly, development of a truly long-acting and stable human insulin analog still remains an important task.

Functional analogs of insulin that may be employed in accordance with the invention include rapid acting analogs such as lispro, aspart and glulisine, which are absorbed rapidly (<30 minutes) after subcutaneous injection, peak at one hour, and have a relatively short duration of action (3 to 4 hours). In addition, two long acting insulin analogs have been developed: glargine and detemir, and which may be employed in connection with the invention. The long acting insulin analogs have an onset of action of approximately two hours and reach a plateau of biological action at 4 to 6 hours, and may last up to 24 hours.

Thus, in one embodiment, the insulin amino acid sequence may contain the A and/or B chain of lispro (also known as HUMALOG, Eli Lilly). Insulin lispro differs from human insulin by the substitution of proline with lysine at position 28 and the substitution of lysine with proline at position 29 of the insulin B chain. Although these modifications do not alter receptor binding, they help to block the formation of insulin dimers and hexamers, allowing for larger amounts of active monomeric insulin to be available for postprandial injections.

In another embodiment, the insulin amino acid sequence may contain an A and/or B chain of aspart (also known as NOVOLOG, Novo Nordisk). Insulin aspart is designed with the single replacement of the amino acid proline by aspartic acid at position 28 of the human insulin B chain. This modification helps block the formation for insulin hexamers, creating a faster acting insulin.

In yet another embodiment, the insulin amino acid sequence may contain an A and/or B chain of glulisine (also known as APIDRA, Sanofi-Aventis). Insulin glulisine is a short acting analog created by substitution of asparagine at position 3 by lysine and lysine at position 29 by glutamine of human insulin B chain. Insulin glulisine has more rapid onset of action and shorter duration of action compared to regular human insulin.

In another embodiment, the insulin amino acid sequence may contain an A and/or B chain of glargine (also known as LANTUS, Sanofi-Aventis). LANTUS has delayed absorption due to its acidic pH that causes microprecipitate formation of insulin crystals in the presence of neutral physiologic pH. Insulin glargine differs from human insulin in that the amino acid asparagine at position 21 of the A chain is replaced by glycine and two arginines are added to the C-terminus of the B-chain. Compared with bedtime neutral protamine Hagedorn (NPH) insulin (an intermediate acting insulin), insulin glargine is associated with less nocturnal hypoglycemia in patients with type 2 diabetes.

In yet another embodiment, the insulin amino acid sequence may contain an A and/or B chain from detemir (also known as LEVEMIR, Novo Nordisk). Insulin detemir is a soluble (at neutral pH) long-acting insulin analog, in which the amino acid threonine at B30 is removed and a 14-carbon, myristoyl fatty acid is acetylated to the epsilon-amino group of LysB29. After subcutaneous injection, detemir dissociates, thereby exposing the free fatty acid which enables reversible binding to albumin molecules. So at steady state, the concentration of free unbound insulin is greatly reduced resulting in stable plasma glucose levels.

In some embodiments, the insulin amino acid sequence may be a single-chain insulin analog (SIA) (e.g. as described in U.S. Pat. No. 6,630,438 and WO 2008/019368, which are hereby incorporated by reference in their entirety). Single-chain insulin analogs encompass a group of structurally-related proteins wherein the A and B chains are covalently linked by a polypeptide linker. The polypeptide linker connects the C-terminus of the B chain to the N-terminus of the A chain. The linker may be of any length so long as the linker provides the structural conformation necessary for the SIA to have a glucose uptake and insulin receptor binding effect. In some embodiments, the linker is about 5-18 amino acids in length. In other embodiments, the linker is about 9-15 amino acids in length. In certain embodiments, the linker is about 12 amino acids long. In certain exemplary embodiments, the linker has the sequence KDDNPNLPRLVR (SEQ ID NO.: 17) or GAGSSSRRAPQT (SEQ ID NO.: 18). However, it should be understood that many variations of this sequence are possible such as in the length (both addition and deletion) and substitutions of amino acids without substantially compromising the effectiveness of the produced SIA in glucose uptake and insulin receptor binding activities. For example, several different amino acid residues may be added or removed from either end without substantially decreasing the activity of the produced SIA.

An exemplary single-chain insulin analog currently in clinical development is albulin (Duttaroy et al., 2005, Diabetes 54: 251-8). Albulin can be produced in yeast or in mammalian cells. It consists of the B and A chain of human insulin (100% identity to native human insulin) linked together by a dodecapeptide linker and fused to the NH2 terminals of the native human serum albumin. For expression and purification of albulin, Duttaroy et al. constructed a synthetic gene construct encoding a single-chain insulin containing the B- and A-chain of mature human insulin linked together by a dodecapeptide linker using four overlapping primers and PCR amplification. The resulting PCR product was ligated in-frame between the signal peptide of human serum albumin (HSA) and the NH2 terminus of mature HSA, contained within a pSAC35 vector for expression in yeast. In accordance with the present invention, the HSA component of abulin may be replaced with an amino acid sequence providing a sustained release as described herein.

Thus, in one aspect, the present invention provides pharmaceutical compositions comprising an amino acid sequence providing a sustained release, including, for example, an elastin-like peptide (ELP), and an insulin amino acid sequence. For example, in certain embodiments, the insulin is a mammalian insulin, such as human insulin or porcine insulin. In accordance with the invention, the amino acid sequence providing a sustained release component may be coupled (e.g., via recombinant fusion or chemical conjugation) to the insulin A chain, or B chain, or both. In some embodiments, the amino acid sequence that provides a slow absorption from the injection site is covalently bound to the insulin A chain. The insulin may comprise each of chains A, B, and C (SEQ ID NOs: 19 and 20), or may contain a processed form, containing only chains A and B. In some embodiments, chains A and B are connected by a short linking peptide, to create a single chain insulin. The insulin may be a functional analog of human insulin, including functional fragments truncated at the N-terminus and/or C-terminus (of either or both of chains A and B) by from 1 to 10 amino acids, including by 1, 2, 3, or about 5 amino acids. Functional analogs may contain from 1 to 10 amino acid insertions, deletions, and/or substitutions (collectively) with respect to the native sequence (e.g., SEQ ID NOs: 15 and 16), and in each case retaining the activity of the peptide. For example, functional analogs may have 1, 2, 3, 4, or 5 amino acid insertions, deletions, and/or substitutions (collectively) with respect to the native sequence (which may contain chains A and B, or chains A, B, and C). Such activity may be confirmed or assayed using any available assay, including those described herein. In these or other embodiments, the insulin component has at least about 75%, 80%, 85%, 90%, 95%, or 98% identity with each of the native sequences for chains A and B (SEQ ID NOs:15 and 16). The determination of sequence identity between two sequences (e.g., between a native sequence and a functional analog) can be accomplished using any alignment tool, including Tatusova et al., Blast 2 sequences—a new tool for comparing protein and nucleotide sequences, FEMS Microbiol Lett. 174:247-250 (1999). The insulin component may contain additional chemical modifications known in the art.

To characterize the in vitro binding properties of an insulin analog or an amino acid sequence providing a sustained release-containing insulin analog, competition binding assays may be performed in various cell lines that express the insulin receptor (Jehle et al., 1996, Diabetologia 39: 421-432). For example, competition binding assays using CHO cells overexpressing the human insulin receptor may be employed. Insulin can also bind to the IGF-1 receptor with a lower affinity than the insulin receptor. To determine the binding affinity of an amino acid sequence providing a sustained release-containing insulin analog, a competition binding assay can be performed using ¹²⁵I-labeled IGF-1 in L6 cells.

The activities of insulin include stimulation of peripheral glucose disposal and inhibition of hepatic glucose production. The ability of an amino acid sequence providing a sustained release-containing insulin analog to mediate these biological activities can be assayed in vitro using known methodologies. For example, the effect of an amino acid sequence providing a sustained release-containing analog on glucose uptake in 3T3-L1 adipocytes can be measured and compared with that of insulin. Pretreatment of the cells with a biologically active analog will generally produce a dose-dependent increase in 2-deoxyglucose uptake. The ability of an amino acid sequence providing a sustained release-containing insulin analog to regulate glucose production may be measured in any number of cells types, for example, H4IIe hepatoma cells. In this assay, pretreatment with a biologically active analog will generally result in a dose-dependent inhibition of the amount of glucose released.

### Amino Acid Sequences Providing Sustained Release

In some embodiments, the amino acid sequence providing sustained release comprises structural units that form hydrogen-bonds through protein backbone groups and/or side chain groups, and which may contribute hydrophobic interactions to matrix formation. In some embodiments, the amino acid side chains do not contain hydrogen bond donor groups, with hydrogen bonds being formed substantially through the protein backbone. Exemplary amino acids include proline, alanine, valine, glycine, and isoleucine, and similar amino acids. In some embodiments, the structural units are substantially repeating structural units, so as to create a substantially repeating structural motif, and substantially repeating hydrogen-bonding capability. In these and other embodiments, the amino acid sequence comprises at least 10%, at least 20%, at least 40%, or at least 50% proline, which may be positioned in a substantially repeating pattern. The substantially repeating pattern of proline may create a repeating β-turn structure. In this context, a substantially repeating pattern means that at least 50% or at least 75% of the proline residues of the amino acid sequence are part of a definable structural unit. In still other embodiments, the amino acid sequence comprises amino acids with hydrogen-bond donor side chains, such as serine, threonine, and/or tyrosine. In some embodiments, the repeating sequence may contain from one to about four proline residues, with remaining residues independently selected from non-polar residues, such as glycine, alanine, leucine, isoleucine, and valine. Non-polar or hydrophobic residues may contribute hydrophobic interactions to the formation of the matrix.

The amino acid sequences may form a “gel-like” state upon injection at a temperature higher than the storage temperature. Exemplary sequences have repeating peptide units, and/or may be relatively unstructured at the lower temperature, and achieve a hydrogen-bonded, structured, state at the higher temperature.

In some embodiments, the amino acid sequence capable of forming the matrix at body temperature is a peptide having repeating units of from four to ten amino acids. The repeating unit may form one, two, or three hydrogen bonds in the formation of the matrix. In certain embodiments, the amino acid sequence capable of forming the matrix at body temperature is an amino acid sequence of silk, elastin, collagen, or keratin, or mimic thereof, or an amino acid sequence disclosed in U.S. Pat. No. 6,355,776, which is hereby incorporated by reference.

In certain embodiments, the amino acid sequence is an Elastin-Like-Protein (ELP) sequence. The ELP sequence comprises or consists of structural peptide units or sequences that are related to, or mimics of, the elastin protein. The ELP sequence is constructed from structural units of from three to about twenty amino acids, or in some embodiments, from four to ten amino acids, such as four, five or six amino acids. The length of the individual structural units may vary or may be uniform. Exemplary structural units include units defined by SEQ ID NOS: 1-12 (below), which may be employed as repeating structural units, including tandem-repeating units, or may be employed in some combination. Thus, the ELP may comprise or consist essentially of structural unit(s) selected from SEQ ID NOS: 1-12, as defined below.

In some embodiments, including embodiments in which the structural units are ELP units, the amino acid sequence comprises or consists essentially of from about 10 to about 500 structural units, or in certain embodiments about 50 to about 200 structural units, or in certain embodiments from about 80 to about 200 structural units, or from about 80 to about 150 structural units, such as one or a combination of units defined by SEQ ID NOS: 1-12. Thus, the structural units collectively may have a length of from about 50 to about 2000 amino acid residues, or from about 100 to about 800 amino acid residues, or from about 200 to about 700 amino acid residues, or from about 400 to about 600 amino acid residues.

The amino acid sequence may exhibit a visible and reversible inverse phase transition with the selected formulation. That is, the amino acid sequence may be structurally disordered and highly soluble in the formulation below a transition temperature (Tt), but exhibit a sharp (2-3° C. range) disorder-to-order phase transition when the temperature of the formulation is raised above the Tt. In addition to temperature, length of the amino acid polymer, amino acid composition, ionic strength, pH, pressure, temperature, selected solvents, presence of organic solutes, and protein concentration may also affect the transition properties, and these may be tailored in the formulation for the desired absorption profile. Absorption profile can be easily tested by determining plasma concentration or activity of the insulin amino acid sequence over time.

In certain embodiments, the ELP component(s) may be formed of structural units, including but not limited to:
(a) the tetrapeptide Val-Pro-Gly-Gly, or VPGG (SEQ ID NO: 1);
(b) the tetrapeptide Ile-Pro-Gly-Gly, or IPGG (SEQ ID NO: 2);
(c) the pentapeptide Val-Pro-Gly-X-Gly (SEQ ID NO: 3), or VPGXG, where X is any natural or non-natural amino acid residue, and where X optionally varies among polymeric or oligomeric repeats;
(d) the pentapeptide Ala-Val-Gly-Val-Pro, or AVGVP (SEQ ID NO: 4);
(e) the pentapeptide Ile-Pro-Gly-X-Gly, or IPGXG (SEQ ID NO: 5), where X is any natural or non-natural amino acid residue, and where X optionally varies among polymeric or oligomeric repeats;
(f) the pentapeptide Ile-Pro-Gly-Val-Gly, or IPGVG (SEQ ID NO: 6);
(g) the pentapeptide Leu-Pro-Gly-X-Gly, or LPGXG (SEQ ID NO: 7), where X is any natural or non-natural amino acid residue, and where X optionally varies among polymeric or oligomeric repeats;
(h) the pentapeptide Leu-Pro-Gly-Val-Gly, or LPGVG (SEQ ID NO: 8);
(i) the hexapeptide Val-Ala-Pro-Gly-Val-Gly, or VAPGVG (SEQ ID NO: 9);
(j) the octapeptide Gly-Val-Gly-Val-Pro-Gly-Val-Gly, or GVGVPGVG (SEQ ID NO: 10);
(k) the nonapeptide Val-Pro-Gly-Phe-Gly-Val-Gly-Ala-Gly, or VPGFGVGAG (SEQ ID NO: 11); and
(l) the nonapeptides Val-Pro-Gly-Val-Gly-Val-Pro-Gly-Gly, or VPGVGVPGG (SEQ ID NO: 12).

Such structural units defined by SEQ ID NOs: 1-12 may form structural repeat units, or may be used in combination to form an ELP. In some embodiments, the ELP component is formed entirely (or almost entirely) of one or a combination of (e.g., 2, 3 or 4) structural units selected from SEQ ID NOs: 1-12. In other embodiments, at least 75%, or at least 80%, or at least 90% of the ELP component is formed from one or a combination of structural units selected from SEQ ID NOs: 1-12, and which may be present as repeating units.

In certain embodiments, the ELP comprises repeat units, including tandem repeating units, of Val-Pro-Gly-X-Gly (SEQ ID NO: 3), where X is as defined above, and where the percentage of Val-Pro-Gly-X-Gly (SEQ ID NO: 3) units taken with respect to the entire ELP component (which may comprise structural units other than VPGXG (SEQ ID NO: 3)) is greater than about 50%, or greater than about 75%, or greater than about 85%, or greater than about 95% of the ELP. The ELP may contain motifs of 5 to 15 structural units (e.g. about 10 structural units) of SEQ ID NO: 3, with the guest residue X varying among at least 2 or at least 3 of the units in the motif. The guest residues may be independently selected, such as from non-polar or hydrophobic residues, such as the amino acids V, I, L, A, G, and W (and may be selected so as to retain a desired inverse phase transition property).

In some embodiments, the ELP may form a β-turn structure. Exemplary peptide sequences suitable for creating a β-turn structure are described in International Patent Application PCT/US96/05186, which is hereby incorporated by reference in its entirety. For example, the fourth residue (X) in the sequence VPGXG (SEQ ID NO: 3), can be altered without eliminating the formation of a β-turn.

The structure of exemplary ELPs may be described using the notation ELPk [XiYj-n], where k designates a particular ELP repeat unit, the bracketed capital letters are single letter amino acid codes and their corresponding subscripts designate the relative ratio of each guest residue X in the structural units (where applicable), and n describes the total length of the ELP in number of the structural repeats. For example, ELP1 [V5A2G3-10] designates an ELP component containing 10 repeating units of the pentapeptide VPGXG (SEQ ID NO: 3), where X is valine, alanine, and glycine at a relative ratio of about 5:2:3; ELP1 [K1V2F1-4] designates an ELP component containing 4 repeating units of the pentapeptide VPGXG (SEQ ID NO: 3), where X is lysine, valine, and phenylalanine at a relative ratio of about 1:2:1; ELP1 [K1V7F1-9] designates a polypeptide containing 9 repeating units of the pentapeptide VPGXG (SEQ ID NO: 3), where X is lysine, valine, and phenylalanine at a relative ratio of about 1:7:1; ELP1 [V-5] designates a polypeptide containing 5 repeating units of the pentapeptide VPGXG (SEQ ID NO:3), where X is valine; ELP1 [V-20] designates a polypeptide containing 20 repeating units of the pentapeptide VPGXG (SEQ ID NO: 3), where X is valine; ELP2 [5] designates a polypeptide containing 5 repeating units of the pentapeptide AVGVP (SEQ ID NO: 4); ELP3 [V-5] designates a polypeptide containing 5 repeating units of the pentapeptide IPGXG (SEQ ID NO: 5), where X is valine; ELP4 [V-5] designates a polypeptide containing 5 repeating units of the pentapeptide LPGXG (SEQ ID NO: 7), where X is valine.

With respect to ELP, the Tt is a function of the hydrophobicity of the guest residue. Thus, by varying the identity of the guest residue(s) and their mole fraction(s), ELPs can be synthesized that exhibit an inverse transition over a broad range. Thus, the Tt at a given ELP length may be decreased by incorporating a larger fraction of hydrophobic guest residues in the ELP sequence. Examples of suitable hydrophobic guest residues include valine, leucine, isoleucine, phenylalanine, tryptophan and methionine. Tyrosine, which is moderately hydrophobic, may also be used. Conversely, the Tt may be increased by incorporating residues, such as those selected from: glutamic acid, cysteine, lysine, aspartate, alanine, asparagine, serine, threonine, glycine, arginine, and glutamine.

For polypeptides having a molecular weight >100,000, the hydrophobicity scale disclosed in PCT/US96/05186 (which is hereby incorporated by reference in its entirety) provides one means for predicting the approximate Tt of a specific ELP sequence. For polypeptides having a molecular weight <100,000, the Tt may be predicted or determined by the following quadratic function: Tt=M0+M1X+M2X2 where X is the MW of the fusion protein, and M0=116.21; M1=-1.7499; M2=0.010349.

The ELP in some embodiments is selected or designed to provide a Tt ranging from about 10 to about 37° C. at formulation conditions, such as from about 20 to about 37° C., or from about 25 to about 37° C. In some embodiments, the transition temperature at physiological conditions (e.g., 0.9% saline) is from about 34 to 36° C., to take into account a slightly lower peripheral temperature.

In certain embodiments, the amino acid sequence capable of forming the hydrogen-bonded matrix at body temperature comprises [VPGXG]₉₀ (SEQ ID NO: 31), where each X is selected from V, G, and A, and wherein the ratio of V:G:A may be about 5:3:2. For example, the amino acid sequence capable of forming the hydrogen-bonded matrix at body temperature may comprise [VPGXG]₁₂₀ (SEQ ID NO: 32), where each X is selected from V, G, and A, and wherein the ratio of V:G:A may be about 5:3:2. As shown herein, 120 structural units of this ELP can provide a transition temperature at about 37° C. with about 5 to 15 mg/ml (e.g., about 10 mg/ml) of protein. At concentrations of about 40 to about 100 mg/mL the phase transition temperature is about 35 degrees centigrade (just below body temperature), which allows for peripheral body temperature to be just less than 37° C.

Alternatively, the amino acid sequence capable of forming the matrix at body temperature comprises [VPGVG]₉₀ (SEQ ID NO: 31), or [VPGVG]₁₂₀ (SEQ ID NO: 32). As shown herein, 120 structural units of this ELP can provide a transition temperature at about 37° C. with about 0.005 to about 0.05 mg/ml (e.g., about 0.01 mg/ml) of protein.

Elastin-like-peptide (ELP) protein polymers and recombinant fusion proteins can be prepared as described in U.S. Patent Publication No. 2010/0022455, which is hereby incorporated by reference.

In other embodiments, the amino acid sequence capable of forming the matrix at body temperature may include a random coil or non-globular extended structure. For example, the amino acid sequence capable of forming the matrix at body temperature may comprise an amino acid sequence disclosed in U.S. Patent Publication No. 2008/0286808, WIPO Patent Publication No. 2008/155134, and U.S. Patent Publication No. 2011/0123487, each of which is hereby incorporated by reference. In some embodiments, the amino acid sequence capable of forming the matrix at body temperature may be predominantly composed of proline with one or more of serine, alanine, and glycine residues. In some embodiments, the amino acid sequence capable of forming the matrix at body temperature is 50%, or 60%, or 70%, or 75%, or 80%, or 90% of proline, serine, alanine, and glycine residues (collectively).

For example, in some embodiments the amino acid sequence comprises an unstructured recombinant polymer of at least 40 amino acids. For example, the unstructured polymer may be defined where the sum of glycine (G), aspartate (D), alanine (A), serine (S), threonine (T), glutamate (E) and proline (P) residues contained in the unstructured polymer, constitutes more than about 80% of the total amino acids. In some embodiments, at least 50% of the amino acids are devoid of secondary structure as determined by the Chou-Fasman algorithm. The unstructured polymer may comprise more than about 100, 150, 200 or more contiguous amino acids. In some embodiments, the amino acid sequence forms a random coil domain. In particular, a polypeptide or amino acid polymer having or forming “random coil conformation” substantially lacks a defined secondary and tertiary structure.

In various embodiments, the intended subject is human, and the body temperature is about 37° C., and thus the pharmaceutical composition is designed to provide a sustained release at this temperature. A slow release into the circulation with reversal of hydrogen bonding and/or hydrophobic interactions is driven by a drop in concentration as the product diffuses at the injection site, even though body temperature remains constant. In other embodiments, the subject is a non-human mammal, and the pharmaceutical composition is designed to exhibit a sustained release at the body temperature of the mammal, which may be from about 30 to about 40° C. in some embodiments, such as for certain domesticated pets (e.g., dog or cat) or livestock (e.g., cow, horse, sheep, or pig). Generally, the Tt is higher than the storage conditions of the formulation (which may be from 10 to about 25° C., or from 15 to 22° C.), such that the pharmaceutical composition remains in solution for injection.

In some embodiments, the slow release is effected by administering cold formulations (e.g. 2-15° C., or 2-10° C., or 2-5° C.) of the pharmaceutical compositions of the present invention. Accordingly, in some embodiments, cold formulations are provided. Cold formulations may be administered at from about 2 to about 3° C., about 2 to about 4° C., about 2 to about 5° C., about 2 to about 6° C., about 2 to about 7° C., about 2 to about 8° C., about 2 to about 10° C., about 2 to about 12° C., about 2 to about 14° C., about 2 to about 15° C., about 2 to about 16° C., about 2 to about 20° C., about 10 to about 25° C., or from 15 to 22° C.

The pharmaceutical composition is generally for “systemic delivery,” meaning that the agent is not delivered locally to a pathological site or a site of action. Instead, the agent is absorbed into the bloodstream from the injection site, where the agent acts systemically or is transported to a site of action via the circulation.

### Sustained Release

In one aspect, the invention provides a sustained release pharmaceutical formulation. The formulation comprises a pharmaceutical composition for systemic administration, where the pharmaceutical composition comprises an insulin amino acid sequence and an amino acid sequence capable of forming a reversible matrix (i.e. an amino acid sequence providing sustained release) at the body temperature of a subject as described herein. The reversible matrix is formed from hydrogen bonds (e.g., intra- and/or intermolecular hydrogen bonds) as well as from hydrophobic contributions. The formulation further comprises one or more pharmaceutically acceptable excipients and/or diluents inducing the formation of the matrix upon administration. The matrix provides for a slow absorption to the circulation from an injection site. The sustained release, or slow absorption from the injection site, is due to a slow reversal of the matrix as the concentration dissipates at the injection site. Once product moves into the circulation, the formulation confers long half-life and improved stability. Thus, a unique combination of slow absorption and long half-life is achieved leading to a desirable PK profile with a shallow peak to trough ratio and/or long Tmax.

Specifically, the invention provides improved pharmacokinetics for peptide drugs like insulin amino acid sequences, including a relatively flat PK profile with a low ratio of peak to trough, and/or a long Tmax. The PK profile can be maintained with a relatively infrequent administration schedule, such as from one to eight injections per month in some embodiments.

In one aspect, the invention provides a sustained release pharmaceutical formulation. The formulation comprises a pharmaceutical composition for systemic administration, where the pharmaceutical composition comprises an insulin amino acid sequence and an amino acid sequence capable of forming a matrix at the body temperature of a subject. The reversible matrix is formed from hydrogen bonds (e.g., intra- and/or intermolecular hydrogen bonds) as well as from hydrophobic contributions. The formulation further comprises one or more pharmaceutically acceptable excipients and/or diluents inducing the formation of the matrix upon administration. The matrix provides for a slow absorption to the circulation from an injection site, and without being bound by theory, this slow absorption is due to the slow reversal of the matrix as protein concentration decreases at the injection site. The slow absorption profile provides for a flat PK profile, as well as convenient and comfortable administration regimen. For example, in various embodiments, the plasma concentration of the insulin amino acid sequence over the course of days (e.g., from 2 to about 60 days, or from about 4 to about 30 days) does not change by more than a factor of 10, or by more than a factor of about 5, or by more than a factor of about 3. Generally, this flat PK profile is seen over a plurality of (substantially evenly spaced) administrations, such as at least 2, at least about 5, or at least about 10 administrations of the formulation. In some embodiments, the slow absorption is exhibited by a Tmax (time to maximum plasma concentration) of greater than about 5 hours, greater than about 10 hours, greater than about 20 hours, greater than about 30 hours, or greater than about 50 hours.

The sustained release, or slow absorption from the injection site, is controlled by the amino acid sequence capable of forming a hydrogen-bonded matrix at the body temperature of the subject, as well as the components of the formulation.

The formulation comprises one or more pharmaceutically acceptable excipients and/or diluents inducing the formation of the matrix upon administration. For example, such excipients include salts, and other excipients that may act to stabilize hydrogen bonding. Exemplary salts include alkaline earth metal salts such as sodium, potassium, and calcium. Counter ions include chloride and phosphate. Exemplary salts include sodium chloride, potassium chloride, magnesium chloride, calcium chloride, and potassium phosphate.

The protein concentration in the formulation is tailored to drive, along with the excipients, the formation of the matrix at the temperature of administration. For example, higher protein concentrations help drive the formation of the matrix, and the protein concentration needed for this purpose varies depending on the ELP series used. For example, in embodiments using an ELP1-120, or amino acid sequences with comparable transition temperatures, the protein is present in the range of about 1 mg/mL to about 200 mg/mL, or is present in the range of about 5 mg/mL to about 125 mg/mL. The pharmaceutical composition may be present in the range of about 10 mg/mL to about 50 mg/mL, or about 15 mg/mL to about 30 mg/mL. In embodiments using an ELP4-120, or amino acid sequences with comparable transition temperatures, the protein is present in the range of about 0.005 mg/mL to about 50 mg/mL, or is present in the range of about 0.01 mg/mL to about 20 mg/mL.

The pharmaceutical composition is formulated at a pH, ionic strength, and generally with excipients sufficient to drive the formation of the matrix at body temperature (e.g., 37° C., or at from 34 to 36° C. in some embodiments). The pharmaceutical composition is generally prepared such that it does not form the matrix at storage conditions. Storage conditions are generally less than the transition temperature of the formulation, such as less than about 32° C., or less than about 30° C., or less than about 27° C., or less than about 25° C., or less than about 20° C., or less than about 15° C. For example, the formulation may be isotonic with blood or have an ionic strength that mimics physiological conditions. For example, the formulation may have an ionic strength of at least that of 25 mM Sodium Chloride, or at least that of 30 mM Sodium chloride, or at least that of 40 mM Sodium Chloride, or at least that of 50 mM Sodium Chloride, or at least that of 75 mM Sodium Chloride, or at least that of 100 mM Sodium Chloride, or at least that of 150 mM Sodium Chloride. In certain embodiments, the formulation has an ionic strength less than that of about 0.9% saline. In some embodiments, the formulation comprises two or more of calcium chloride, magnesium chloride, potassium chloride, potassium phosphate monobasic, sodium chloride, and sodium phosphate dibasic.

In certain embodiments, the formulation may comprise about 50 mM histidine, or about 40 mM histidine, or about 30 mM histidine, or about 25 mM histidine, or about 20 mM histidine, or about 15 mM histidine.

The liquid formulation may comprise about 100 mM Sodium Chloride and about 20 mM histidine and can be stored refrigerated or at room temperature. The salt concentration can be altered to provide isotonicity at the site of injection.

The formulation can be packaged in the form of pre-dosed pens or syringes for administration once per week, twice per week, or from one to eight times per month, or alternatively filled in conventional vial and the like.

In exemplary embodiments, the invention provides a sustained release pharmaceutical formulation that comprises a therapeutic agent, the therapeutic agent (e.g., a peptide or protein therapeutic agent) comprising an insulin amino acid sequence and an amino acid sequence comprising [VPGXG]₉₀ (SEQ ID NO: 31), or [VPGXG]₁₂₀ (SEQ ID NO: 32), where each X is selected from V, G, and A. V, G, and A may be present at a ratio of about 5:3:2. Alternatively, the amino acid sequence comprises [VPGVG]₉₀ (SEQ ID NO: 31) or [VPGVG]₁₂₀ (SEQ ID NO: 32). The formulation further comprises one or more pharmaceutically acceptable excipients and/or diluents for formation of a reversible matrix from an aqueous form upon administration to a human subject. Insulin and derivatives thereof are described herein and in U.S. Provisional Application No. 61/563,985, which is hereby incorporated by reference

In these embodiments, the insulin amino acid sequence may be present in the range of about 0.5 mg/mL to about 200 mg/mL, or is present in the range of about 5 mg/mL to about 125 mg/mL. The insulin amino acid sequence is present in the range of about 10 mg/mL to about 50 mg/mL, or the range of about 15 mg/mL to about 30 mg/mL The formulation may have an ionic strength of at least that of 25 mM Sodium Chloride, or at least that of 30 mM sodium Chloride, or at least that of 40 mM Sodium Chloride, or at that least that of 50 mM Sodium Chloride, or at least that of 75 mM Sodium Chloride, or at least that of 100 mM Sodium Chloride. The formulation may have an ionic strength less than that of about 0.9% saline. The formulation comprises two or more of calcium chloride, magnesium chloride, potassium chloride, potassium phosphate monobasic, sodium chloride, and sodium phosphate dibasic.

Other formulation components for achieving the desired stability, for example, may also be employed. Such components include one or more amino acids or sugar alcohol (e.g., mannitol), preservatives, and buffering agents, and such ingredients are well known in the art.

In another aspect, the invention provides a method for delivering a sustained release regimen of an insulin amino acid sequence. The method comprises administering the formulation described herein to a subject in need, wherein the formulation is administered from about 1 to about 8 times per month.

In some embodiments, the formulation is administered about weekly, and may be administered subcutaneously or intramuscularly. In some embodiments, the site of administration is not a pathological site, for example, is not the intended site of action.

In various embodiments, the plasma concentration of the insulin amino acid sequence does not change by more than a factor of 10, or a factor of about 5, or a factor of about 3 over the course of a plurality of administrations, such as at least 2, at least about 5, or at least about 10 administrations of the formulation. The administrations are substantially evenly spaced, such as, for example, about daily, or about once per week, or from one to about five times per month.

In certain embodiments, the subject is a human, but in other embodiments may be a non-human mammal, such as a domesticated pet (e.g., dog or cat), or livestock or farm animal (e.g., horse, cow, sheep, or pig).

### Conjugation and Coupling

A recombinantly-produced fusion protein, in accordance with certain embodiments of the invention, includes an amino acid sequence providing sustained release (e.g., ELP) and an insulin amino acid sequence associated with one another by genetic fusion. For example, the fusion protein may be generated by translation of a polynucleotide encoding an insulin amino acid sequence cloned in-frame with the amino acid sequence providing sustained release component.

In certain embodiments, the amino acid sequence providing sustained release component and insulin amino acid sequence can be fused using a linker peptide of various lengths to provide greater physical separation and allow more spatial mobility between the fused portions, and thus maximize the accessibility of the insulin amino acid sequence for binding to its receptor. The linker peptide may consist of amino acids that are flexible or more rigid. For example, a flexible linker may include amino acids having relatively small side chains, and which may be hydrophilic. Without limitation, the flexible linker may comprise glycine and/or serine residues. More rigid linkers may contain, for example, more sterically hindering amino acid side chains, such as (without limitation) tyrosine or histidine. The linker may be less than about 50, 40, 30, 20, 10, or 5 amino acid residues. The linker can be covalently linked to and between an insulin amino acid sequence and an amino acid sequence providing sustained release component, for example, via recombinant fusion.

The linker or peptide spacer may be protease-cleavable or non-cleavable. By way of example, cleavable peptide spacers include, without limitation, a peptide sequence recognized by proteases (in vitro or in vivo) of varying type, such as Tev, thrombin, factor Xa, plasmin (blood proteases), metalloproteases, cathepsins (e.g., GFLG, SEQ ID NO: 21, etc.), and proteases found in other corporeal compartments. In some embodiments employing cleavable linkers, the fusion protein may be inactive, less active, or less potent as a fusion, which is then activated upon cleavage of the spacer in vivo. Alternatively, where the insulin amino acid sequence is sufficiently active as a fusion, a non-cleavable spacer may be employed. The non-cleavable spacer may be of any suitable type, including, for example, non-cleavable spacer moieties having the formula [(Gly)n-Ser]m (SEQ ID NO: 34), where n is from 1 to 4, inclusive, and m is from 1 to 4, inclusive. Alternatively, a short ELP sequence different than the backbone ELP could be employed instead of a linker or spacer, while accomplishing the necessary effect.

In still other embodiments, the pharmaceutical composition is a recombinant fusion having a insulin amino acid sequence flanked on each terminus by an amino acid sequence providing sustained release component. At least one of the amino acid sequence providing sustained release components may be attached via a cleavable spacer, such that the insulin amino acid sequence is inactive, but activated in vivo by proteolytic removal of a single ELP component. The resulting single amino acid sequence providing sustained release fusion being active, and having an enhanced half-life (or other property described herein) in vivo.

In other embodiments, the present invention provides chemical conjugates of an insulin amino acid sequence and the amino acid sequence providing sustained release component. The conjugates can be made by chemically coupling an amino acid sequence providing sustained release component to an insulin amino acid sequence by any number of methods well known in the art (See, e.g., Nilsson et al., 2005, Ann Rev Biophys Bio Structure 34: 91-118). In some embodiments, the chemical conjugate can be formed by covalently linking the insulin amino acid sequence to the amino acid sequence providing sustained release component, directly or through a short or long linker moiety, through one or more functional groups on the therapeutic proteinacious component, e.g., amine, carboxyl, phenyl, thiol or hydroxyl groups, to form a covalent conjugate. Various conventional linkers can be used, e.g., diisocyanates, diisothiocyanates, carbodiimides, bis(hydroxysuccinimide) esters, maleimide-hydroxysuccinimide esters, glutaraldehyde and the like.

Non-peptide chemical spacers can additionally be of any suitable type, including for example, by functional linkers described in Bioconjugate Techniques, Greg T. Hermanson, published by Academic Press, Inc., 1995, and those specified in the Cross-Linking Reagents Technical Handbook, available from Pierce Biotechnology, Inc. (Rockford, Ill.), the disclosures of which are hereby incorporated by reference, in their respective entireties. Illustrative chemical spacers include homobifunctional linkers that can attach to amine groups of Lys, as well as heterobifunctional linkers that can attach to Cys at one terminus, and to Lys at the other terminus.

In certain embodiments, relatively small ELP components (e.g., ELP components of less than about 30 kDa, 25 kDa, 20 kDa, 15 kDa, or 10 kDa), that do not transition at room temperature (or human body temperature, e.g., Tt>37° C.), are chemically coupled or crosslinked. For example, two relatively small ELP components, having the same or different properties, may be chemically coupled. Such coupling, in some embodiments, may take place in vivo, by the addition of a single cysteine residue at or around the C-terminus of the ELP. Such ELP components may each be fused to one or more insulin amino acid sequences, so as to increase activity or avidity at the target.

### Methods of Treating Diseases

In various embodiments, the pharmaceutical compositions of the present invention as described herein are used for the management and care of a patient having a pathology such as diabetes or hyperglycemia, or any other condition for which insulin administration is indicated for the purpose of combating or alleviating symptoms and complications of those conditions, including various metabolic disorders. Treating includes administering a formulation of present invention to prevent the onset of the symptoms or complications, alleviating the symptoms or complications, or eliminating the disease, condition, or disorder. The present methods include treatment of type 1 diabetes, i.e., a condition in which the body does not produce insulin and therefore cannot control the amount of sugar in the blood and type 2 diabetes, i.e., a condition in which the body does not use insulin normally and, therefore, cannot control the amount of sugar in the blood.

In various embodiments, the sustained release provides for sustained glycemic control Glycemic control refers to the typical levels of blood sugar (glucose) in a person with diabetes mellitus. Many of the long-term complications of diabetes, including microvascular complications, result from many years of hyperglycemia. Good glycemic control is an important goal of diabetes care. Because blood sugar levels fluctuate throughout the day and glucose records are imperfect indicators of these changes, the percentage of hemoglobin which is glycosylated is used as a proxy measure of long-term glycemic control in research trials and clinical care of people with diabetes. In this test, the hemoglobin A1c or glycosylated hemoglobin reflects average glucose values over the preceding 2-3 months.

In nondiabetic persons with normal glucose metabolism glycosylated hemoglobin levels are usually about 4-6% by the most common methods (normal ranges may vary by method). “Perfect glycemic control” indicates that glucose levels are always normal (e.g. about 70-130 mg/dl, or about 3.9-7.2 mmol/L) and indistinguishable from a person without diabetes. In reality, because of the imperfections of treatment measures, even “good glycemic control” describes blood glucose levels that average somewhat higher than normal much of the time. It is noted that what is considered “good glycemic control” varies by age and susceptibility of the patient to hypoglycemia. The American Diabetes Association has advocated for patients and physicians to strive for average glucose and hemoglobin A1c values below 200 mg/dl (11 mmol/1) and 8%. “Poor glycemic control” refers to persistently elevated blood glucose and glycosylated hemoglobin levels, which may range from, e.g., about 200-500 mg/dl (about 11-28 mmol/L) and about 9-15% or higher over months and years before severe complications occur.

In various embodiments, the present invention provides for combination therapies and/or co-formulations which comprise the pharmaceutical compositions described herein and other agents that are effective in treating diseases, such as those described above.

In one embodiment, the invention provides for combination or co-formulation with glucagon like receptor (GLP)-1 receptor agonist, such as GLP-1 (SEQ ID NO: 22), exendin-4 (SEQ ID NO: 23), or functional analogs and/or derivatives thereof as disclosed in U.S. Pat. No. 8,178,495, which is hereby incorporated by reference. In some embodiments, the GLP-1 is GLP-1 (A-B), wherein A is an integer from 1 to 7 and B is an integer from 38 to 45. In some embodiments, the GLP-1 is GLP-1 (7-36) (SEQ ID NO: 24), or a functional analog thereof or GLP-1 (7-37) (SEQ ID NO: 25), or a functional analog thereof.

In another embodiment, the invention provides for combination or co-formulation with GLP-2 (SEQ ID NO: 26), GIP (SEQ ID NO: 27), glucagon (SEQ ID NO: 28), and oxyntomodulin (SEQ ID NO: 29) or functional analogs and/or derivatives thereof. Functional analogs may contain from 1 to 10 amino acid insertions, deletions, and/or substitutions (collectively) with respect to the native sequence.

In various embodiments, the combination therapies and/or co-formulations comprise fusion proteins with, for example, ELP or a matrix-forming component as described herein. In some embodiments, the ELP comprises at least 60 units (SEQ ID NO: 30), or 90 units (SEQ ID NO: 31), or 120 units (SEQ ID NO: 32), or 180 units of VPGXG (SEQ ID NO: 33), where X is an independently selected amino acid. In various embodiments, X is V, G, or A at a ratio of 5:3:2, or K, V, or F at a ratio of 1:2:1, or K, V, or F at a ratio of 1:7:1, or V.

In another embodiment, the invention provides for combination or co-formulation with various forms of insulin as described herein. In one embodiment, the insulin is a fast, or rapid, acting insulin.

### EXAMPLES

Human proinsulin was genetically fused to the ELP1-120 biopolymer and expressed in the soluble fraction of E coli. Following purification enzymatic processing of the proinsulin moiety into mature insulin the fusion protein was tested for glucose lowering in a normal mouse model and compared with insulin alone. The insulin ELP fusion showed glucose lowering similar to insulin. In addition the lowering effect of the fusion protein was shown to extend over a longer duration than that of insulin in the model.

### Insulin Fusion Construction

Human proinsulin consists of the B and A chains linked together with the 31 amino acid C peptide (FIGS. 1A and 1B). Once disulfides are formed between the B and A chains the proinsulin is converted into mature insulin in vivo by removal of the C peptide by a trypsin/carboxypeptidase B-like system. This peptide processing can be replicated in vitro using recombinant trypsin and carboxypeptidase B. Since the fusion is expressed in the soluble fraction of E. coli no refolding steps are necessary.

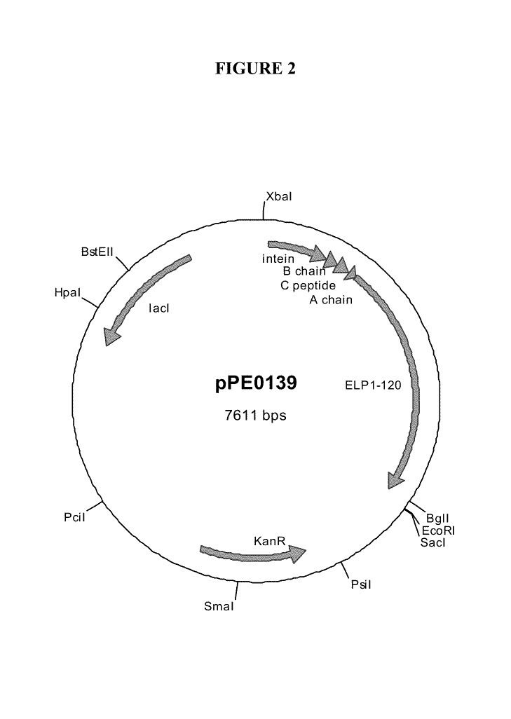

The proinsulin nucleotide sequence was synthesized and subcloned into pET based vector pPB1031 positioning it at the N-terminus of the ELP1-120 sequence to make plasmid pPE0139 (FIG. 2).

FIG. 3 shows the amino acid sequence of a proinsulin ELP1-120 fusion protein (SEQ ID NO: 14). The proinsulin sequence (underlined) is fused to the ELP1-120 sequence. The amino acid sequence optionally includes an initiation methionine residue at the N terminus.

### Fermentation

Insulin ELP fusion plasmid pPE0139 was expressed in the intracellular fraction of E. coli under control of the T7 promoter in a fed-batch fermentation process. The glycerol cell stock was expanded using a two-stage shake flask seed train in semi-defined, animal-free medium (ECPM+Proline) with glycerol as the primary carbon source and yeast extract as the primary nitrogen source. After sufficient cell density was achieved in the seed train, the culture was transferred to a fermentor containing the same medium as the seed train. Process parameters (pH, temperature, dissolved oxygen) were maintained at set point via PID control. The culture grew until it reached stationary phase whereupon a glycerol/yeast extract/magnesium sulfate feed was initiated. The culture was maintained under carbon limitation and induction of the promoter was achieved using IPTG. At the end of the fermentation, the culture was centrifuged to separate the biomass containing the Insulin ELP fusion from the spent medium. The cell paste was stored at −70° C. until subsequent purification.

### Purification

Frozen cell paste was resuspended in lysis buffer containing 2M Urea (for dissociation of Insulin ELP) and mixed until homogenous. Lysis was achieved using a microfluidizer to disrupt the cell membranes. A two stage tangential flow filtration (TFF) system was used to clarify and concentrate the product. The Insulin ELP fusion was passed over a HIC column as a capture step and host cell contaminants were washed away. The product was eluted using a gradient to fractionate any product-related impurities (degraded species). TFF buffer exchange was performed on the selected fractions to remove residual salt prior to two anion exchange column to remove residual DNA, endotoxin and host cell proteins. A final TFF concentration and buffer exchange was used to formulate the product. 0.2 μM filtration was used for sterilization. The product was stored at 4° C. until enzymatic digestion.

### Enzymatic Processing of Proinsulin ELP Fusion (PE0083)

Purified Proinsulin ELP1-120 was diluted to 1 mg/mL in formulation buffer. A 2× enzyme solution for processing of Proinsulin ELP into mature Insulin ELP was prepared as follows: 50 mM Sodium Bicarbonate, 2 ug/mL trypsin and 20 ug/mL carboxypeptidase B. The 2× enzyme solution was added to an equal volume of 1 mg/mL PE0083 and incubated at 37° C. for 1-2 hours. The enzymatic reaction was stopped using the phase transition properties of ELP. Sodium chloride was added to the reaction to induce phase transitioning of the fusion. The mature Insulin ELP formed a coacervate and was pelleted via centrifugation. The residual enzymes were washed away and the pelleted fusion was resolubilized in a low salt buffer. Two phase transition purifications were performed.

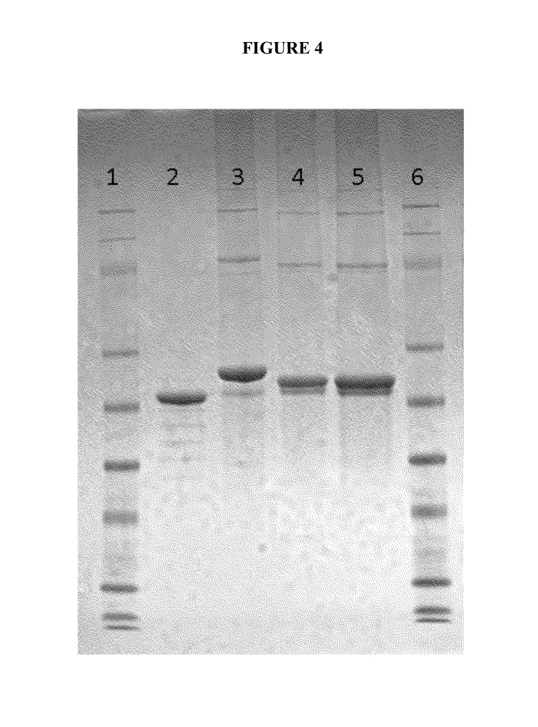

Non-reducing SDS-PAGE (FIG. 4) showed the expected decreased fusion protein molecular weight following enzymatic processing as the C-peptide was cleaved.

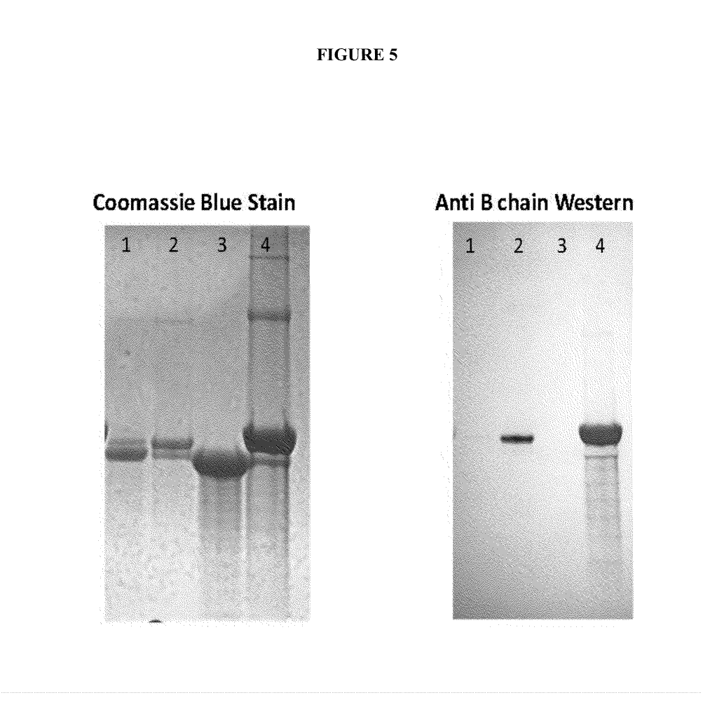

An anti-insulin B chain western blot (FIG. 5) was performed to confirm presence of both A and B chains fused to ELP. The data showed presence of B-chain under non-reducing conditions indicating disulfide bond formation between the A and B chains. Reduction of the fusion protein and disulfide bonds resulted in removal of B chain from the fusion.

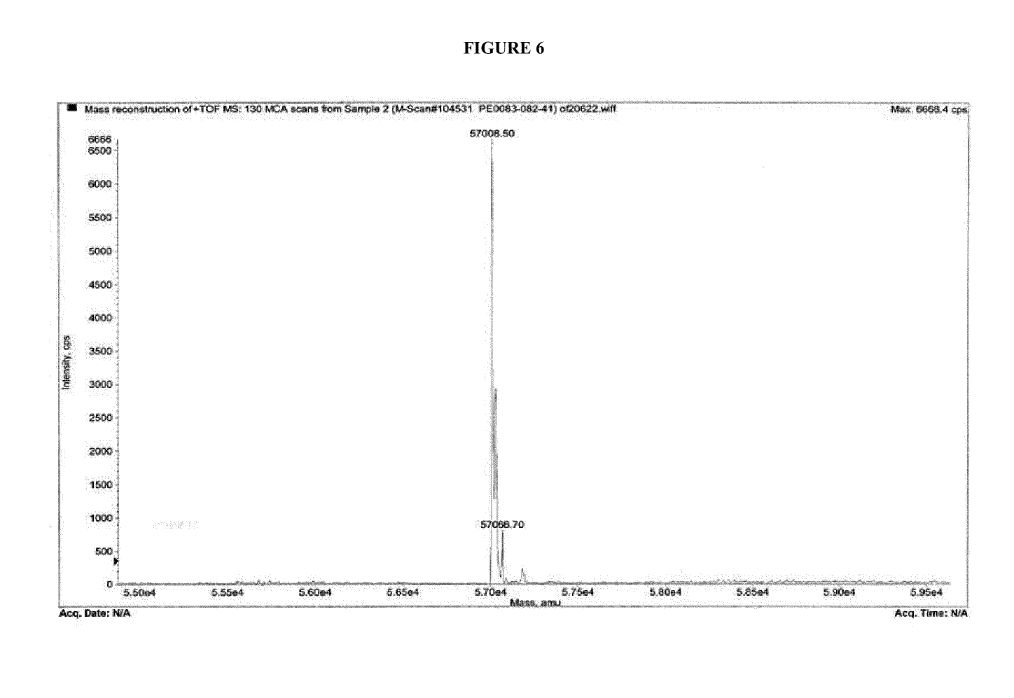

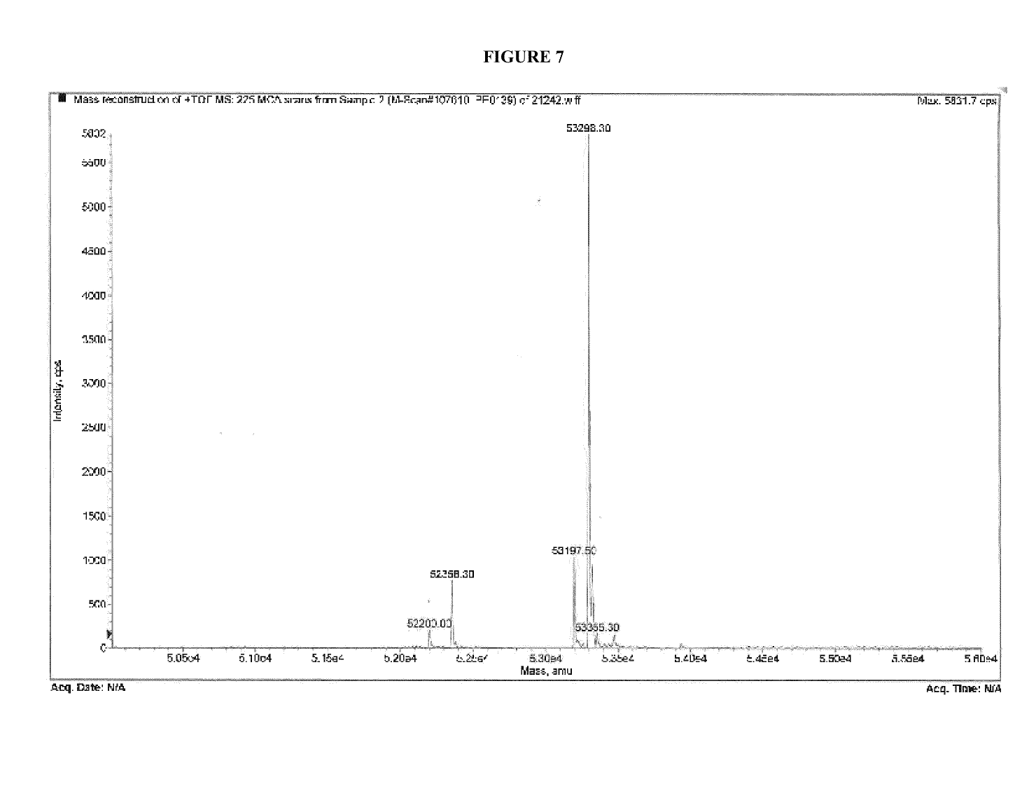

Electrospray ionization mass spectrometry confirmed the mass of Proinsulin ELP fusion (FIG. 6) and the mature Insulin ELP fusion following enzymatic removal of the C-peptide (FIG. 7). Additional salt adducts were present in both samples. Presence of disulfide bonds was confirmed using an Ellman's reagent assay. The absence of free thiols indicated disulfide bonds were formed.

In vivo Glucose Lowering

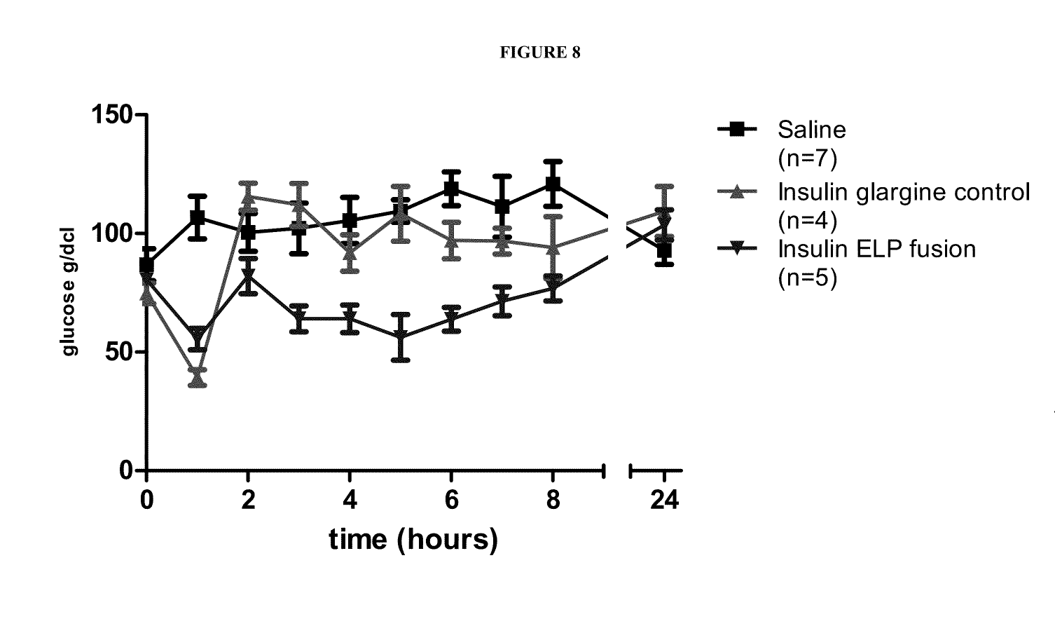

Normal mice were fasted overnight and injected subcutaneously with saline (negative control), 13 nmol/kg insulin glargine (positive control) or 35 nmol/kg insulin ELP fusion (INSUMERA). Blood glucose readings were taken prior to dosing and each hour after through 8 hours and 24 hours post dosing. Food was made available 1 hour post dose. FIG. 8 shows the blood glucose data (mean+−SE). The insulin ELP fusion shows significant blood glucose lowering versus the saline control. In addition the insulin ELP fusion showed a blood glucose lowering that extended farther (7 hours) than the insulin glargine control (2 hours).

### In Vivo Effects in a Type 1 Diabetes Model

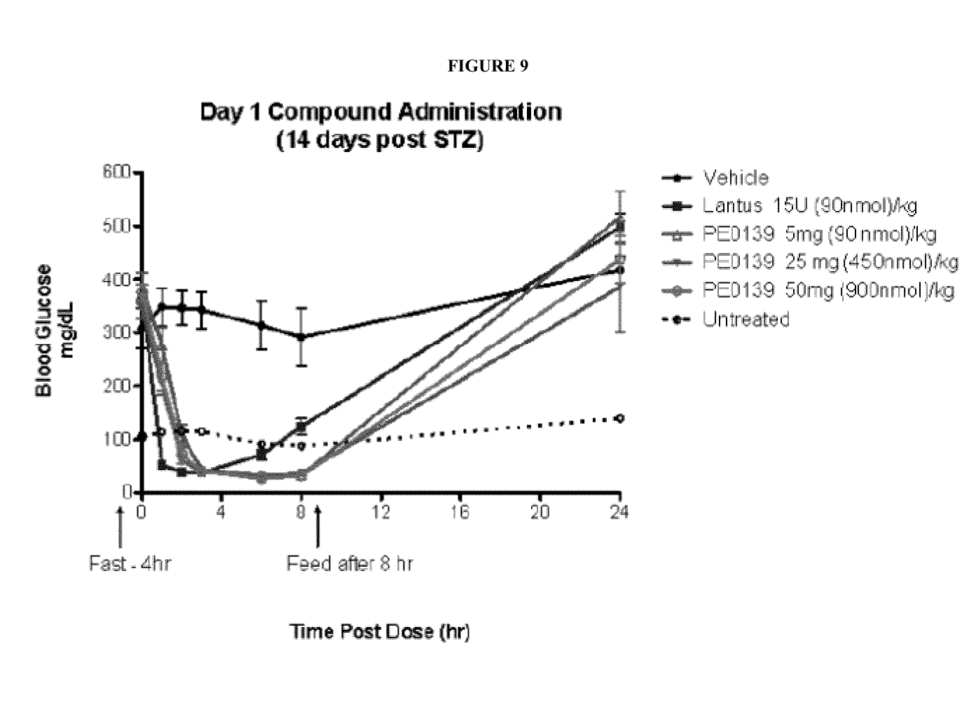

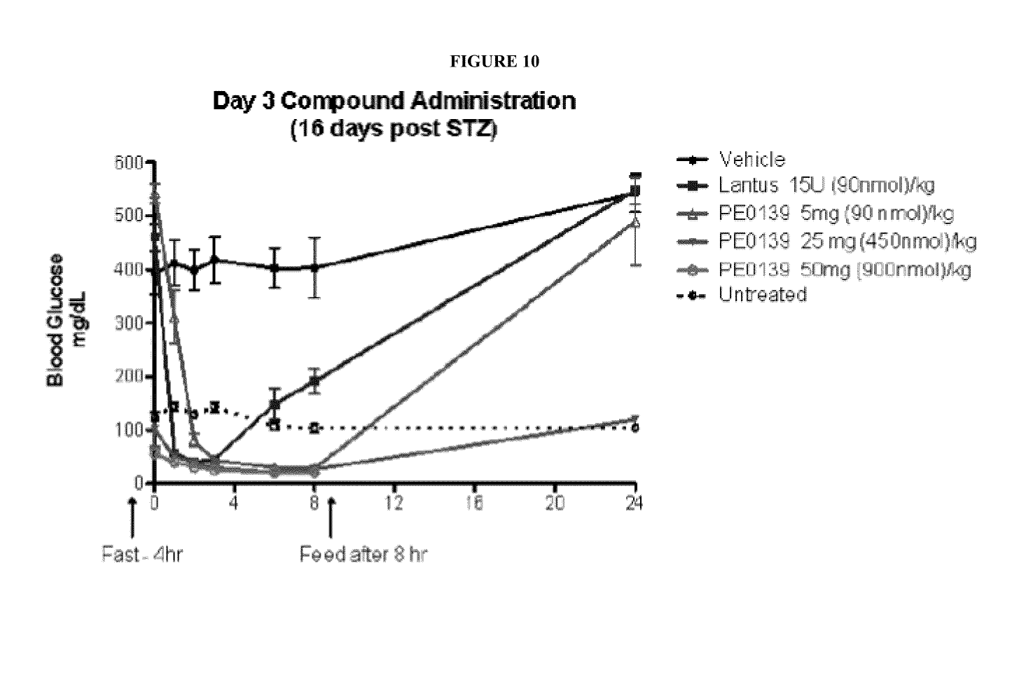

A ELP-Insulin fusion, INSUMERA (PE0139), was dosed in a diabetes mellitus type 1 (type 1 diabetes, T1DM) mouse model. Specifically, single dose data is shown in FIG. 9. The results demonstrated a greater duration of glucose lowering for INSUMERA, as compared to equimolar LANTUS (insulin glargine, SANOFI-AVENTIS) dosing. When the compounds were dosed on a daily regimen (FIG. 10), the results demonstrate the superiority of INSUMERA, as compared to LANTUS (insulin glargine, SANOFI-AVENTIS), with regards to activity and haft-life.

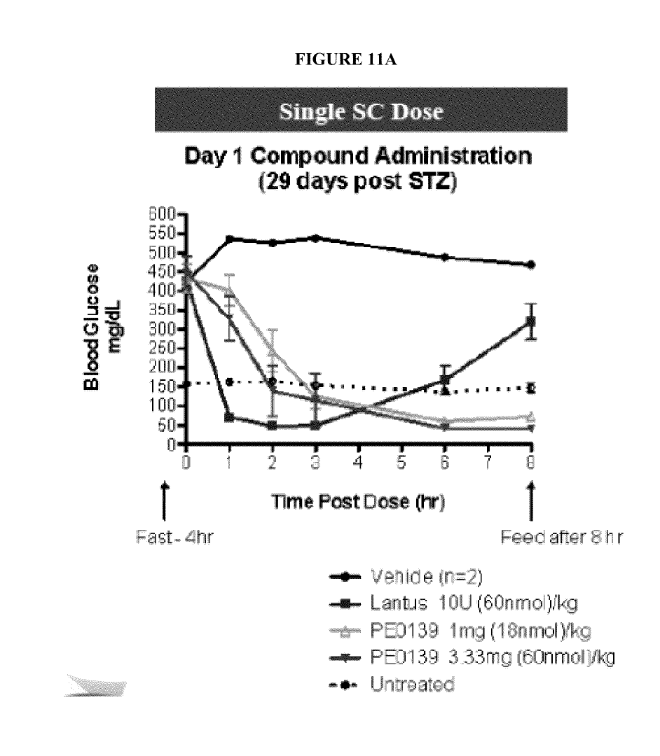

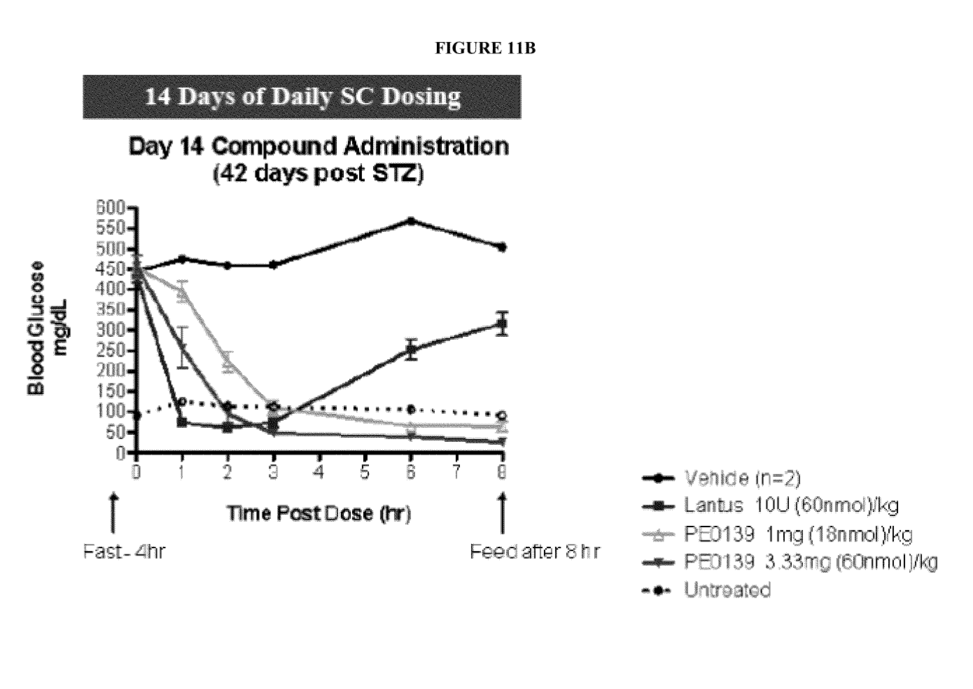

FIGS. 11A and 11B show INSUMERA (PEO139) low dose titration in a diabetes mellitus type 1 (type 1 diabetes, T1DM) mouse model as compared to LANTUS (insulin glargine, SANOFI-AVENTIS). FIG. 11A shows a single s.c. dose while FIG. 11B shows 14 days of daily s.c. dosing. In both cases, the more pronounced and sustained blood glucose lowering effect of INSUMERA is shown.

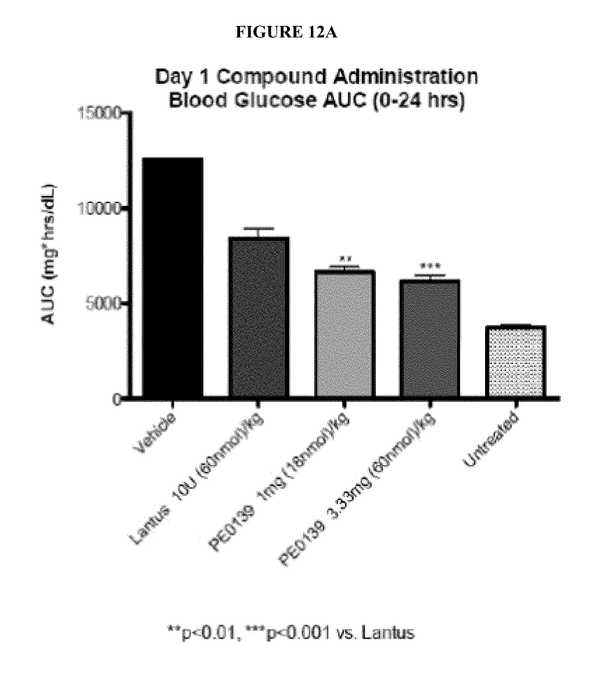

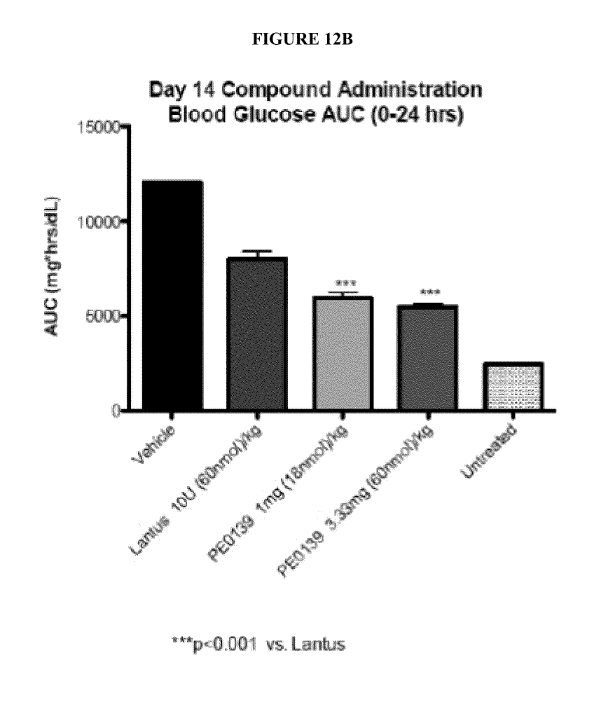

Studies were also conducted to determine the extent of glycemic control, a measure of the typical levels of blood sugar in a patient, of INSUMERA. FIGS. 12A and 11B shows that INSUMERA (PE0139) has significantly increased glycemic control relative to LANTUS (insulin glargine, SANOFI-AVENTIS). A reduction of 27-39% is seen in area under the curve (AUC) blood glucose. FIG. 12A shows day 1 of compound administration and the blood glucose AUC at 0-24 hrs. FIG. 12B shows day 14 of compound administration and the blood glucose AUC at 0-24 hrs. INSUMERA reduced blood glucose AUC, more effectively than LANTUS, in both dosing regimes.

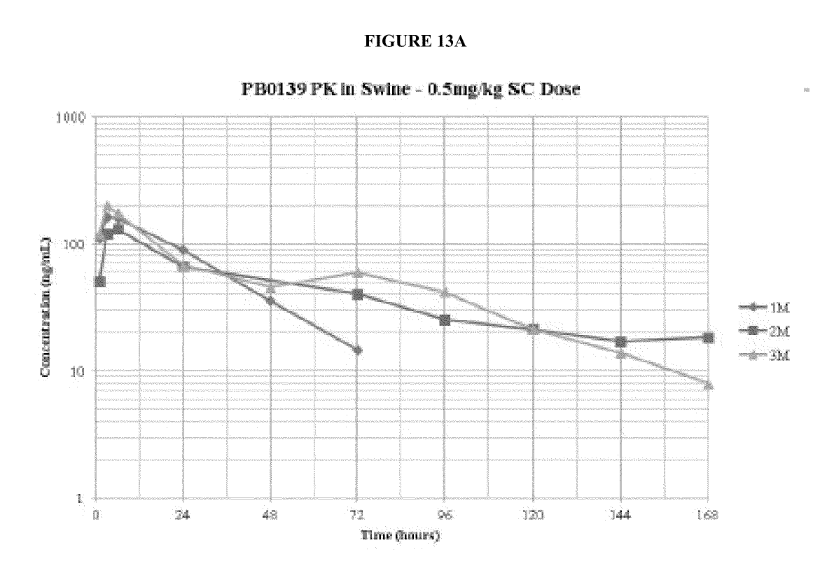

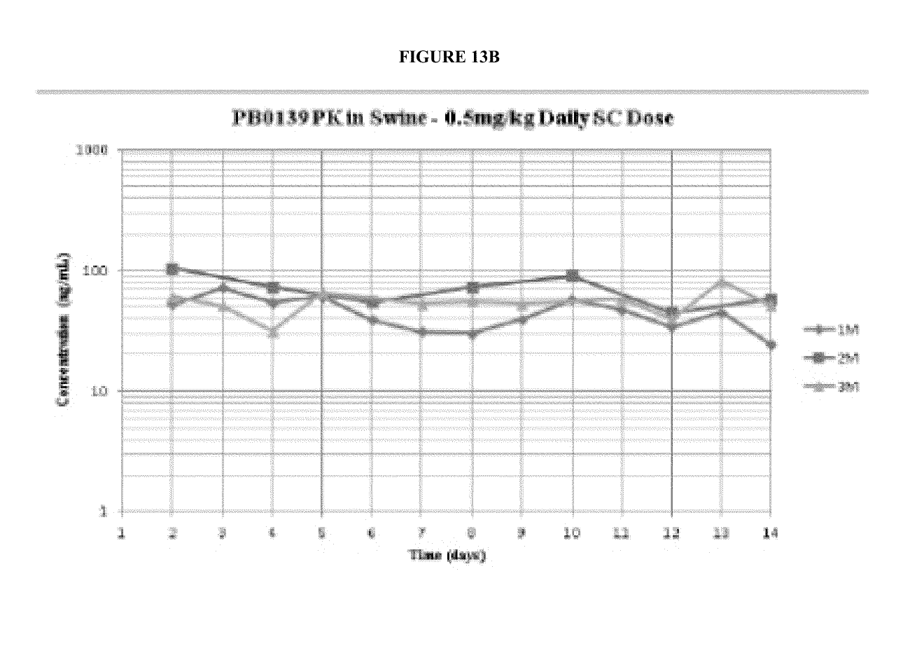

Studies were also conducted to evaluate the pharmacokinetics (PK) of INSUMERA treatment. In diabetic swine, either a single s.c. injection (FIG. 13A) or daily s.c. injections for 2 weeks (FIG. 13B) regimen was followed. The results show that INSUMERA achieves a long half-life with a small peak to trough ratio following a subcutaneous injection.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A pharmaceutical composition for providing sustained glycemic control, comprising an effective amount of a protein comprising an insulin amino acid sequence and an amino acid sequence providing a sustained release from an injection site, and pharmaceutical excipients to achieve sustained release.

2. The pharmaceutical composition of claim 1, wherein the insulin amino acid sequence comprises an A chain and a B chain amino acid sequence, wherein the A chain and B chain have the amino acid sequence of SEQ ID NO: 13, optionally having from 1 to 8 amino acid insertions, deletions, or substitutions, collectively.

3. The pharmaceutical composition of claim 2, wherein the amino acid sequence that provides a slow absorption from the injection site is covalently bound to the insulin A chain.

4. The pharmaceutical composition of claim 2 or 3, wherein the A chain and B chain are bound by one or more disulfide bonds.

5. The pharmaceutical composition of claim 2 or 3, wherein the A chain and B chain are attached through a peptide or chemical linker.

6. (canceled)

7. The pharmaceutical composition of claim 1, wherein the amino acid sequence providing a sustained release is an elastin-like peptide (ELP) amino acid sequence.

8. The pharmaceutical composition of claim 7, wherein the ELP comprises repeats of VPGXG (SEQ ID NO: 3), where each X is independently selected from alanine, arginine, asparagine, aspartic acid, glutamic acid, glutamine, glycine, histidine, isoleucine, leucine, lysine, methionine, phenylalanine, serine, threonine, tryptophan, tyrosine and valine residues.

9. (canceled)

10. The pharmaceutical composition of claim 7, wherein the amino acid sequence of the ELP comprises repeats of AVGVP (SEQ ID NO: 4), IPGVG (SEQ ID NO: 6), or LPGVG (SEQ ID NO: 8).

11. (canceled)

12. (canceled)

13. The pharmaceutical composition of claim 8, wherein the ELP comprises at least 60 repeats of an ELP amino acid unit.

14. (canceled)

15. (canceled)

16. (canceled)

17. The pharmaceutical composition of claim 1, wherein the pharmaceutical composition comprises SEQ ID NO: 14.

18. The pharmaceutical composition of claim 7, wherein the ELP has a transition temperature of just less than 37° C. in normal saline.

19. (canceled)

20. (canceled)

21. The pharmaceutical composition of claim 1, wherein the pharmaceutical composition is a fusion protein.

22. The pharmaceutical composition of claim 1, wherein the pharmaceutical composition comprises about 110 mM sodium chloride and about 20 mM histidine.

23. The pharmaceutical composition of claim 1, wherein the pharmaceutical composition is formulated for administration from 1 to about 30 times per month.

24. (canceled)

25. The pharmaceutical composition of claim 1, wherein the pharmaceutical composition is formulated for administration at a dosage of about 0.5 mg/mL to about 200 mg/mL.

26. (canceled)

27. A method of treating diabetes comprising administering a pharmaceutical composition for providing sustained glycemic control, comprising an effective amount of a protein comprising an insulin amino acid sequence and an amino acid sequence providing a sustained release from an injection site, and pharmaceutical excipients to achieve sustained release to a patient in need thereof.

28. The method of claim 27, wherein the patient has type 1 diabetes or type 2 diabetes.

29. (canceled)

30. The method of claim 27, wherein the pharmaceutical composition is administered at a frequency of from 1 to about 30 times per month.

31. (canceled)

32. (canceled)

33. (canceled)

34. The method of claim 27, wherein the pharmaceutical composition is administered subcutaneously.
